(19) United States
(12) Patent Application Publication
(10) Pub. No.: US 2023/0278882 A1
(43) Pub. Date: Sep. 7, 2023

(54) METHOD FOR PRODUCING CALCIUM CARBONATE BY UTILIZING SEA WATER AND BURNED SHELLS, AND CALCIUM CARBONATE AND CALCIUM AGENT PRODUCED THEREBY

(71) Applicant: KOREA MARITIME UNIVERSITY INDUSTRY-ACADEMIC COOPERATION FOUNDATION, Busan (KR)

(72) Inventors: Myoung Jin KIM, Busan (KR); Geun Young KIM, Busan (KR); Se Hun KIM, Seoul (KR); Seon Mi SHIN, Busan (KR)

(73) Assignee: KOREA MARITIME UNIVERSITY INDUSTRY-ACADEMIC COOPERATION FOUNDATION, Busan (KR)

(21) Appl. No.: 18/016,588

(22) PCT Filed: Aug. 10, 2021
(86) PCT No.: PCT/KR2021/010546

§ 371 Date: Jan. 17, 2023

(30) Foreign Application Priority Data
Sep. 7, 2020 (KR) 10-2020-0113731

(51) Int. Cl. C01F 11/18; A23L 33/16

(52) U.S. Cl. CPC C01F 11/183; A23L 33/16; C01P 2002/72; C01P 2002/82; C01P 2004/03; C01P 2004/62

(57) ABSTRACT

There is provided a method for producing calcium carbonate by utilizing seawater and calcinated shells, and calcium carbonate and a calcium agent produced thereby. The method for producing calcium carbonate includes: eluting calcium by mixing calcinated shells, seawater, and sugar; and generating calcium carbonate by injecting carbon dioxide into the calcium eluate generated in the eluting calcium. The calcium agent includes vaterite-type calcium carbonate.

### CROSS-REFERENCE TO PRIOR APPLICATIONS

This application is a National Stage Patent Application of PCT International Patent Application No. PCT/KR2021/010546 (filed on Aug. 10, 2021) under 35 U.S.C. §371, which claims priority to Korean Patent Application No. 10-2020-0113731 (filed on Sep. 7, 2020), which are all hereby incorporated by reference in their entirety.

### BACKGROUND

The present invention relates to a method for producing calcium carbonate utilizing seawater and calcinated shells, as well to calcium carbonate and a calcium agent produced by the method, and more particularly, to a method for producing calcium carbonate utilizing sugar, as one of indirect carbonation methods utilizing seawater and calcinated shells, in particular, to a method for producing vaterite calcium carbonate.

Calcium is a mineral nutrient that is essential for maintaining physiological functions of living organisms. Most of calcium is the basis for the formation of skeleton and teeth in the body, and a small amount of calcium is present in an ionized state, and is actively used for various reactions in the body, such as regulation of the movement of substances through cell membranes, blood coagulation, muscle contraction and relaxation, secretion of neurotransmitters and activation of enzymes. As such, in order to sufficiently ingest calcium which is essential for maintaining life activities, calcium agents have been developed for the purpose of supplementing calcium that is insufficient in daily meals, and many studies are being conducted to achieve better calcium absorption rate.

However, most of the calcium carbonate-based calcium agents on the market today are micro-sized, and because of the large particle size of calcium carbonate, ionization in the stomach is difficult, and the absorption rate in the body is lower than that of the organic acid calcium-based calcium agents. Furthermore, most calcium carbonate in the nature exists in the form of calcite, which forms a stable structure and has low reactivity and solubility, resulting in a lower absorption rate when compared to the crystal phase of other calcium carbonates. In order to overcome this problem, a method of pulverizing calcium carbonate into nano-sized particles is used, however, this method has the disadvantage of requiring a large amount of energy during the pulverization process, which adds to the cost of manufacturing a calcium carbonate-based calcium agent.

Therefore, there is an emerging need for a method for producing a calcium agent with a high absorption rate in the body for having a small calcium carbonate particle size, unlike the conventional calcium carbonate-based calcium agent, and the method having an economical process for producing the same.

### SUMMARY

One purpose of the present invention is to provide a method for producing a nano-sized calcium agent, which exhibits a better absorption rate in the body than that of a conventional micro-sized calcium agent.

One purpose of the present invention is to provide a method for producing a calcium agent of a vaterite crystalline form, which exhibits a better absorption rate in the body than that of a conventional calcium agent of a calcite crystalline form.

One purpose of the present invention is to provide a production method capable of economically producing a calcium agent because an additional calcium carbonate pulverization process is not required.

According to one aspect of the present invention, the present invention provides a method for producing calcium carbonate, more specifically, a method for producing vaterite calcium carbonate, the method comprising: a first step of eluting calcium by mixing calcinated shells, seawater, and sugar; and a second step of producing calcium carbonate by injecting carbon dioxide into the calcium eluate produced in the first step.

The present invention relates to a method for producing calcium carbonate by using calcinated shells containing calcium oxide and seawater containing magnesium ions, wherein the method is distinguished in that the addition of sugar increases the content of vaterite type calcium carbonate, reduces the particle size of vaterite, and increases production when compared to a method that does not involve sugar.

In general, when seawater and shell cause an elution reaction, the following reactions occur by the large amount of salt in seawater.

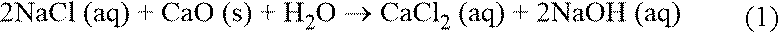

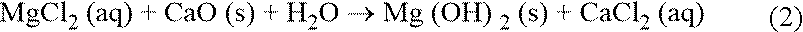

However, the present invention involves a reaction in which calcinated shells, seawater, and sugar are mixed to elute calcium. In contrast to the general reaction, the sugar component acts as a chelating agent, and together with calcium, forms a calcium-sucrose complex (when sucrose is used), which can be represented by the following formulas.

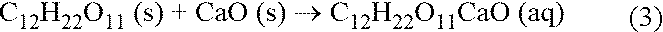

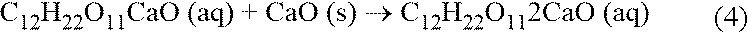

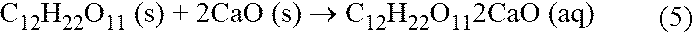

As described above, more calcium can be eluted from shells by the complex action of the sugar component and the large amount of salt in seawater (Equation 1-5), and depending on the solid-liquid ratio (amount of sugar added) of sugar and seawater, the calcium concentration and pH of the eluate can be adjusted more extensively.

Through the principle described above, calcium to be used in the second step of the present invention can be obtained in an ionic state.

The calcium elution reaction of the first step can be expressed by Formula (6):

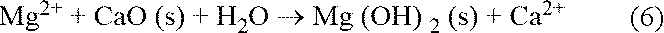

The magnesium ion of Formula (6) is derived from magnesium chloride (MgCl₂) which is present in the form of an ion in the seawater used in the reaction of the first step.

The calcium oxide of Formula (6), which is derived from the calcinated shell, a component formed by calcining the shell containing calcium carbonate as a main component.

Calcium in the shell exists in the form of calcium carbonate, but after the shell is calcined, calcium is extracted through seawater as ionized calcium and a carbonation reaction is carried out by injecting carbon dioxide to resynthesize into a porous nano-sized calcium carbonate of vaterite crystalline form having excellent solubility and absorption rate. As a result, of the nano size and porosity, the calcium carbonate of the present invention has a large surface area and high solubility and dispersity due to the vaterite phase.

When the magnesium ion is subject to a reaction with calcium oxide, a magnesium precipitation reaction occurs and precipitation occurred in the form of Mg(OH)₂, eluting calcium, wherein calcium has an ionic form of Ca²⁺.

The sugar used in the first step does not directly participate in the reaction between magnesium ion and calcium oxide, but plays an important role in the production of the calcium carbonate of the present invention. First, the sugar increases the amount of calcium ion eluted in the first step. Second, the sugar helps to increase the vaterite content of calcium carbonate. Third, the sugar allows for the control of the vaterite particle size. Finally, the sugar allows for the omission of the process of adjusting the pH before the carbonation reaction of the second step. For the four reasons described above, the addition of an appropriate amount of sugar in the method for producing calcium carbonate of the present invention has a critical meaning in the present invention.

The second step is a step of producing calcium carbonate by injecting carbon dioxide into the calcium eluate produced in the first step, wherein calcium carbonate, the final product of the production method of the present invention, is obtained through a carbonation reaction using the calcium eluate and carbon dioxide.

The carbonation reaction in the second step can be expressed by the following formulas (7, 8, 9):

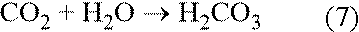

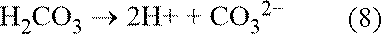

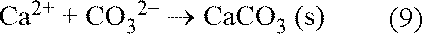

The calcium ions are included in the calcium eluate obtained in the first step, and calcium carbonate was produced through a carbonation reaction in which carbon dioxide was added thereto.

In general, precipitated calcium carbonate as used in the present invention can be prepared by a reaction of a gas and a liquid or by a reaction of a liquid and another liquid. In the present invention, calcium carbonate was produced by a reaction of calcium ions dissolved in a liquid with carbon dioxide gas.

According to one embodiment, the present invention is characterized in that when the solid-liquid ratio of the sugar and seawater added in the first step is 1:80 (g:mL) or less, the vaterite content of calcium carbonate is 100%.

As described above, the sugar added during the first step of the present invention increases the vaterite content of calcium carbonate and aids in the control of vaterite particle size. Unless sugar is added in the first step, the vaterite content of calcium carbonate decreases and the vaterite particle size increases. This is a factor that directly inhibits the effects of the present invention, which are the dissolution of calcium carbonate and the absorption of calcium into the body that are significantly high compared to the prior arts.

In general, calcium carbonate can have three crystal structures, which are calcite, aragonite and vaterite, wherein calcium carbonate that exists in the nature is mostly in the stable calcite form. The vaterite crystalline form used in the present invention has the most unstable crystalline structure among the three crystalline forms of calcium carbonate, and is a porous material with a large specific surface area. For this reason, the vaterite crystalline form has the highest solubility among the three crystalline forms of calcium carbonate, and therefore, when prepared as a calcium agent, it exhibits a high absorption rate in the body.

According to one embodiment, the present invention is characterized in that the sugar used in the production method is sucrose.

According to one embodiment, the present invention is characterized in that when the solid-liquid ratio of the sugar and seawater added in the first step is 1:5000 to 1:500, the vaterite crystal has a particle size of 600 to 800 nm.

The inventor of the present invention found that, when the solid-liquid ratio of the sugar and seawater added in the first step is not between 1:5000 and 1:500, the content of calcite crystalline calcium carbonate increases or the particle size of the calcium carbonate increases, and as a result, the solubility of the calcium carbonate of the present invention is lowered. Therefore, in pursuit of an increase of the calcium elution, an increase of the vaterite crystallization of calcium carbonate, and a decrease of the particle size, sugar should be added in an amount that is not excessive.

According to one embodiment, the present invention is characterized in that the pH after the completion of the first step is 12.5 or higher.

As mentioned above, the reaction in the second step is a carbonation reaction. In the carbonation reaction, when carbon dioxide is added to the calcium ion eluate from the first step, the pH of the calcium eluate is lowered by the carbon dioxide. However, since calcium carbonate, which is a product of the above reaction, is dissolved under acidic conditions, an appropriate pH that is not excessively low is required to obtain calcium carbonate crystals. Therefore, in consideration of the pH decrease by the carbonation reaction, before starting the carbonation reaction of the second step, it is necessary to maintain the pH of the calcium eluate high to exhibit some degree of alkalinity. However, when calcium is eluted by mixing sugar as in the first step, the pH is high enough to omit the process of raising the pH. When no sugar was added in the first step, the calcium eluate had a pH of 12 or lower, but pH of the calcium eluate after adding the sugar was 12.5 or higher, which was a sufficiently high pH to omit the process of raising the pH.

According to one embodiment, the present invention is characterized in that the second step comprises applying ultrasonic waves to the solution to which carbon dioxide has been injected.

According to one embodiment, the present invention is characterized in that the production method further comprises a step of stirring the produced calcium carbonate at room temperature after the carbonation reaction of the second step.

The step of stirring is a step of aging the produced calcium carbonate, and is characterized in that the stirring is performed at 200 rpm.

According to one embodiment, the present invention is characterized in that the step of stirring is performed for 60 minutes or less, preferably for 2 minutes to 20 minutes, and more preferably for 10 minutes.

According to one embodiment, the present invention is characterized in that the particle size of the calcium carbonate produced by the production method is in a range of 600 nm to 800 nm.

According to one embodiment, the present invention is characterized in that the calcium carbonate produced by the production method has porosity.

In addition, according to one aspect of the present invention, the present invention provides a calcium agent comprising vaterite-type calcium carbonate.

According to one embodiment, the present invention provides a calcium agent comprising vaterite-type calcium carbonate produced by a method for producing calcium carbonate, the method comprising: a first step of eluting calcium by mixing calcinated shells, seawater, and sugar; and a second step of producing calcium carbonate by injecting carbon dioxide into the calcium eluate produced in the first step.

According to the present invention, nano-sized fine vaterite calcium agent can be produced, wherein the nano-sized vaterite calcium agent exhibits a better absorption rate in the body compared to the existing micro-sized calcite crystalline calcium agents and thus is capable of overcoming the limit of absorption rate in the body of calcium agents of the exiting calcium carbonate by drastically increasing the degree of ionization of calcium carbonate.

In addition, since the method for producing a fine vaterite calcium agent of the present invention is a production method performed by resynthesis of calcium carbonate instead of a process of pulverizing particles, an additional calcium carbonate pulverization process is not required. Therefore, a calcium agent can be produced more economically compared to the existing method for producing a calcium carbonate-based calcium agent.

### BRIEF DESCRIPTION OF THE DRAWINGS

FIG. 1 shows a schematic flowchart of the method for producing calcium carbonate recited in the present invention.

FIG. 2 shows a schematic diagram of a reactor for the carbonation reaction of the second step.

FIG. 3 shows an XRD graph of calcium carbonate with respect to the amount of sugar added in the first step.

FIG. 4 shows an FT-IR graph of calcium carbonate with respect to the amount of sugar added in the first step.

FIG. 5 shows an SEM image of calcium carbonate particles produced when the amount of sugar added in the first step was 2.34 mM.

FIG. 6 shows a graph about the vaterite particle size change with respect to the stirring speed, and a graph about the vaterite particle size change with respect to the ultrasonic intensity, respectively.

FIG. 7 shows a graph about the vaterite particle size change with respect to the stirring speed and the ultrasonic intensity when stirring and ultrasonic waves were used at the same time.

FIG. 8 shows a table representing the particle size of calcium carbonate produced under different aging conditions after carbonation.

FIG. 9 shows a graph of the relative activity of ALP with respect to the calcium carbonate particle size and crystalline form depending on the amount of injected calcium carbonate.

FIG. 10 shows a graph about the solubility with respect to the calcium carbonate particle size and crystalline form depending on the pH of the surrounding environment.

### DETAILED DESCRIPTION

Hereinafter, Examples of the present invention will be described in detail. Since the present invention can have various changes and can have various forms, specific embodiments are illustrated in the drawings and described in detail in the text. However, the Examples are not intended to limit the present invention to the specific forms of disclosure, and they should be understood to include all modifications, equivalents and substitutes included in the principles and technical scope of the present invention.

Terms such as first, second and so on may be used to describe various features, but the features should not be limited by the terms. The terms are used only for the purpose of distinguishing one feature from another.

Throughout the specification, when a part “includes” or “contains” a certain feature, it means that other features may be further included unless otherwise defined. In addition, the singular expression used in the present Specification includes the plural expression unless the context clearly dictates otherwise.

Unless otherwise defined, all terms used herein, including technical or scientific terms, have the same meaning as commonly understood by one of ordinary skill in the art to which the present invention belongs, and unless explicitly defined in the present application, the terms are not understood in an ideal or overly formal sense.

Hereinafter, the method for producing calcium carbonate utilizing seawater and calcinated shells recited in the present invention, in particular, the method for producing vaterite calcium carbonate, will be described in more detail with reference to the drawings of the present invention.

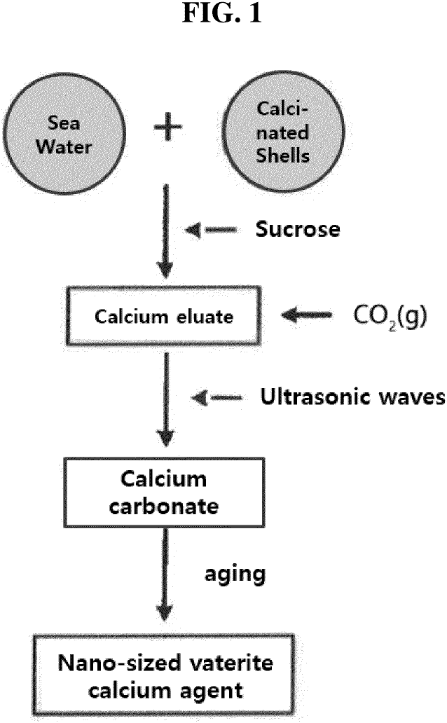

FIG. 1 shows a schematic flowchart of the method for producing calcium carbonate recited in the present invention, in particular, the method for producing vaterite calcium carbonate.

As shown in FIG. 1, the method for producing calcium carbonate of the present invention comprises a first step of eluting calcium by mixing calcinated shells, seawater, and sugar; and a second step of producing calcium carbonate by injecting carbon dioxide into the calcium eluate produced in the first step.

The shell of the first step is a source of calcium carbonate which is a raw material of the manufacturing method, and as the types of the shell, shells of oysters, mussels, shellfish, short-neck clams, or abalones may be used.

The sugar in the first step may be sucrose, glucose, lactose, starch, or fructose, preferably, sucrose. In the manufacturing method of an Example for the experimental demonstration of the present invention, sucrose was treated in the first step.

The amount of sugar added in the first step is 0.58 mM to 5.84 mM, preferably, 2.34 mM. In an Example of the present invention, when the amount of added sucrose was 2.34 mM, the crystal form of calcium carbonate was 100% vaterite, and the size of the synthesized particles was 683 nm.

The pH after completion of the first step is preferably 12.5 or higher.

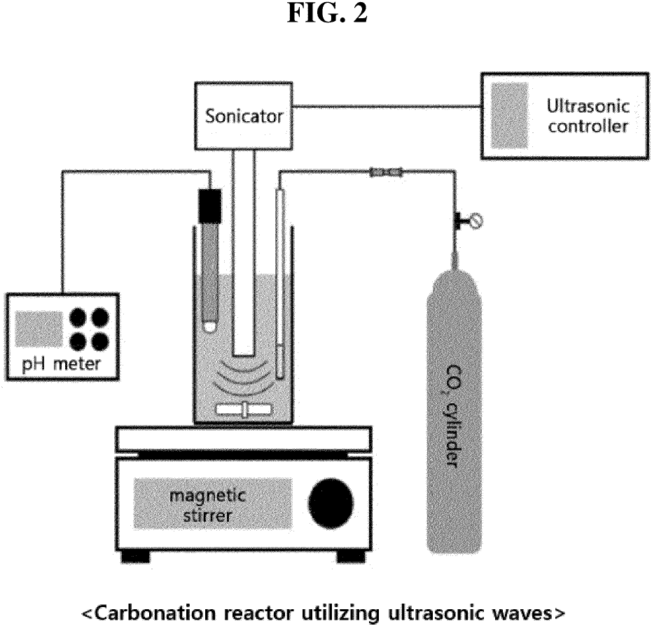

FIG. 2 below shows a schematic diagram of a reactor in which carbon dioxide is injected, ultrasonic waves are applied, stirring is performed, and pH is measured for the carbonation reaction of the second step.

To reduce the particle size of vaterite produced through the carbonation reaction of the second step, ultrasonic waves were additionally applied during the carbonation reaction of the second step.

The carbon dioxide injection was discontinued, and the produced calcium carbonate was stirred at 200 rpm for 60 minutes, preferably, for 10 minutes.

The calcium carbonate obtained from the process above may have a particle size in a range of 600 nm to 800 nm, preferably, 683 nm.

As shown in FIG. 5 below, the calcium carbonate obtained from the process above may have porosity through which the calcium carbonate of the present invention may have high solubility and absorption rate.

Additionally, the calcium carbonate obtained from the process above can be used as a calcium agent.

A test of the effective ingredient of the calcium agent was conducted by measuring the relative activity of ALP with respect to the calcium carbonate particle size depending on the amount of injected calcium carbonate, and by measuring the solubility according to the calcium carbonate particle size depending on the pH of the surrounding environment. The test is described more specifically in the Examples and Evaluation Examples below.

### EXAMPLES

### Examples Depending on the Sugar Content

In order to investigate the change of calcium carbonate depending on the amount of the sugar added in the first step, Examples 1 to 12 and Comparative Examples were prepared as described below by adding sucrose in different amounts.

### Example 1: Solid-Liquid Ratio of Sugar and Seawater 1:5000 (g:mL)

In 100 mL of seawater, sucrose was mixed and dissolved so that the solid-liquid ratio of sugar and seawater became 1:5000, and then the seawater in which sucrose was dissolved and the calcinated CaO were mixed so that the solid-liquid ratio became 1:50. After that, the resulting mixture was stirred at 25° C. at 200 rpm for 1 hour, and then filtered through a 0.45 µm membrane filter (MCE04547A, HYUNDAI Micro Co.).

The calcium eluate filtered through the process above was poured into a beaker and stirred at 400 rpm using a stirrer (HS-30D, WISD), while 99.9% carbon dioxide was injected at a flow rate of 0.15 L/min by a gas disperser (Sigma). A gas flow meter and a flow regulator (TSM-D220, MKP) were used to control the flow rate to be constant, and when ultrasound waves were required, ultrasound waves were applied at a 30% intensity by using a Branson SFX 550 model with a ¼ diameter tip. Ultrasound waves were applied in advance before injecting carbon dioxide. The carbonation reaction was stopped by discontinuing the injection of carbon dioxide gas, and aging was performed by stirring at 200 rpm for 10 minutes. The generated solid was filtered through a 0.1 µm membrane filter (A010A047A, Toyo Roshi Kaisha) and dried at 60° C. for 4 hours.

### Example 2: Solid-Liquid Ratio of Sugar and Seawater 1:2500 (g:mL)

Calcium carbonate was produced in the same manner as in Example 1, except that sucrose was added at a solid-liquid ratio of 1:2500 between sugar and seawater.

### Example 3: Solid-Liquid Ratio of Sugar and Seawater 1:1250 (g:mL)

Calcium carbonate was produced in the same manner as in Example 1, except that sucrose was added at a solid-liquid ratio of 1:1250 between sugar and seawater.

### Example 4: Solid-Liquid Ratio of Sugar and Seawater 1:625 (g:mL)

Calcium carbonate was produced in the same manner as in Example 1, except that sucrose was added at a solid-liquid ratio of 1:625 between sugar and seawater.

### Example 5: Solid-Liquid Ratio of Sugar and Seawater 1:312 (g:mL)

Calcium carbonate was produced in the same manner as in Example 1, except that sucrose was added at a solid-liquid ratio of 1:312 between sugar and seawater.

### Example 6: Solid-Liquid Ratio of Sugar and Seawater 1:156 (g:mL)

Calcium carbonate was produced in the same manner as in Example 1, except that sucrose was added at a solid-liquid ratio of 1:156 between sugar and seawater.

### Example 7: Solid-Liquid Ratio of Sugar and Seawater 1:78 (g:mL)

Calcium carbonate was produced in the same manner as in Example 1, except that sucrose was added at a solid-liquid ratio of 1:78 between sugar and seawater.

### Example 8: Solid-Liquid Ratio of Sugar and Seawater 1:39 (g:mL)

Calcium carbonate was produced in the same manner as in Example 1, except that sucrose was added at a solid-liquid ratio of 1:39 between sugar and seawater.

### Example 9: Solid-Liquid Ratio of Sugar and Seawater 1:27 (g:mL)

Calcium carbonate was produced in the same manner as in Example 1, except that sucrose was added at a solid-liquid ratio of 1:27 between sugar and seawater.

### Example 10: Solid-Liquid Ratio of Sugar and Seawater 1:19 (g:mL)

Calcium carbonate was produced in the same manner as in Example 1, except that sucrose was added at a solid-liquid ratio of 1:19 between sugar and seawater.

### Example 11: Solid-Liquid Ratio of Sugar and Seawater 1:14 (g:mL)

Calcium carbonate was produced in the same manner as in Example 1, except that sucrose was added at a solid-liquid ratio of 1:14 between sugar and seawater.

### Comparative Example: No Sucrose Added

Calcium carbonate was produced in the same manner as in Example 1, except that no sucrose was added.

### EVALUATION EXAMPLE

### 1. Change of Calcium Carbonate Depending on the Production Method

The calcium concentration was measured using an atomic absorption spectrophotometer (AAS, AA200, Perkin Elmer), and the pH was measured by using a pH meter (Orion star 211, Thermo).

In addition, the particle size of calcium carbonate was measured using a laser scattering particle size analyzer (Mastersizer 3000, Malvern).

### Change of the Amount of Calcium Elution Depending on the Sugar Content

First, in order to investigate the total calcium concentration of the calcium eluate after the first step, experiments were prepared in an environment of various solid-liquid ratios between sugars and seawater. As the amount of added sucrose was increased, the concentration of calcium was increased. When the amount of added sucrose was the highest, the calcium concentration was 7125 mg/L, and when no sucrose was added, it was 3100 mg/L.

Table 1 below shows the pH and calcium concentration of the calcium eluate according to the change of the solid-liquid ratio between sugar and seawater in the first step.

Changes in the pH and calcium concentration of the calcium eluate according to the change in the ratio between sucrose and seawater

TABLE 1

| | Sucrose: seawater (g:mL) | Amount of added sucrose (mM) | pH | Total calcium concentration (mg/L) |
|---|---|---|---|---|
| Comparative Example | 0 | 0 | 11.7 | 3100 |
| Example 1 | 1:5000 | 0.58 | 12.5 | 3402 |
| Example 2 | 1:2500 | 1.17 | 12.6 | 3750 |
| Example 3 | 1:1250 | 2.34 | 12.7 | 4000 |
| Example 4 | 1:625 | 4.67 | 12.7 | 4025 |
| Example 5 | 1:312 | 9.35 | 12.9 | 4100 |
| Example 6 | 1:156 | 18.70 | 12.9 | 4842 |
| Example 7 | 1:78 | 37.51 | 12.6 | 5675 |
| Example 8 | 1:39 | 75.02 | 12.5 | 6390 |
| Example 9 | 1:27 | 107.48 | 12.5 | 6425 |
| Example 10 | 1:19 | 150.04 | 12.5 | 6503 |
| Example 11 | 1:14 | 214.99 | 12.4 | 7125 |

As shown in Table 1 above, the pH of the calcium eluate was increased and then decreased as the amount of added sucrose was increased. When the solid-liquid ratio between sugar and seawater was 1:312 or less, as the amount of added sucrose was increased, more calcium sources were dissolved and thereby increasing the pH. When the solid-liquid ratio between sucrose and seawater was 1:312, calcium particles were dissolved in the aqueous sucrose solution, and so the pH of the calcium eluate was the highest, 12.9.

### Change of the Crystalline Form of Calcium Carbonate Depending on the Sugar Content

In order to investigate the change of calcium carbonate depending on the amount of sugar added in the first step, X-ray diffraction analysis (XRD, Smart lab, Rigaku) and Fourier transform infrared spectroscopy (FTIR, Thermo Fisher, iS50) were performed.

Table 2 below shows the changes in the size and shape of the produced calcium carbonate particles according to the change in the amount of added sucrose.

Change of the size and shape of the produced calcium carbonate particles according to the change in the amount of added sucrose.

TABLE 2

| | Sucrose: seawater (g: mL) | Amount of added sucrose (mM) | Median of particle size (D50, um) | Type of CaCO₃ | |
|---|---|---|---|---|---|
| | | | | Vaterite (%) | Calcite (%) |
| Comparative Example | 0 | 0 | 0.870 | 100 | 0 |
| Example 1 | 1:5000 | 0.58 | 0.765 | 100 | 0 |
| Example 2 | 1:2500 | 1.17 | 0.732 | 100 | 0 |
| Example 3 | 1:1250 | 2.34 | 0.683 | 100 | 0 |
| Example 4 | 1:625 | 4.67 | 0.759 | 100 | 0 |
| Example 5 | 1:312 | 9.35 | 0.816 | 100 | 0 |
| Example 6 | 1:156 | 18.70 | 0.844 | 100 | 0 |
| Example 7 | 1:78 | 37.51 | 0.927 | 100 | 0 |
| Example 8 | 1:39 | 75.02 | 0.965 | 97 | 3 |
| Example 9 | 1:27 | 107.48 | 1.07 | 97 | 3 |
| Example 10 | 1:19 | 150.04 | 1.09 | 96 | 4 |
| Example 11 | 1:14 | 214.99 | 1.27 | 92 | 8 |

As shown in Table 2, the particle size of the produced calcium carbonate was the smallest when the solid-liquid ratio between sugar and seawater was 1:1250, that is, when the amount of added sugar was 2.34 mM. In addition, when the solid-liquid ratio between sucrose and seawater was 1:80 or lower, 100% vaterite was produced, whereas when it was higher than that, some calcite was produced.

In addition, sucrose was added to calcium carbonate as in (a) Example 11, (b) Example 10, (c) Example 9, (d) Example 8, (e) Example 7, or (f) Example 3, respectively, to analyze the difference of the calcium carbonate depending on the sugar added in the first step.

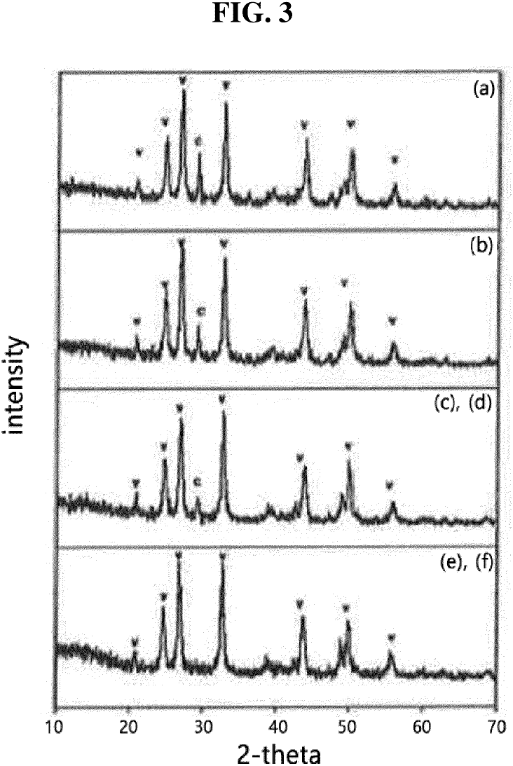

FIG. 3 depicts an XRD graph of the calcium carbonate with respect to the amount of sugar added in the first step. In Examples 8-11 where sucrose was added in amounts of (a) 215 mM, (b) 150 mM, (c) 107 mM, or (d) 75 mM, a calcite peak (indicated by C at the top of the peak) was found between the vaterite peaks (indicated by V at the top of the peak). On the other hand, in Example 7 ((e) 38 mM) or Example 3 ((f) 2.34 mM) where a smaller amount of sucrose was added, no calcite peak was observed on the XRD graph.

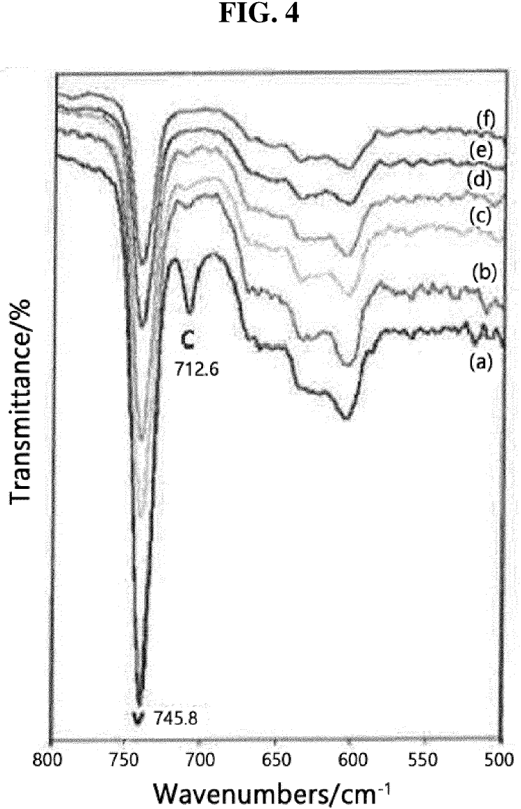

In addition, FIG. 4 shows an FT-IR graph of the calcium carbonate with respect to the amount of sugar added in the first step. FT-IR analysis was performed by using the same samples as in FIG. 3, and the results showed that the calcite peak (indicated by C at the bottom of the peak) disappeared as the amount of added sucrose was reduced as in FIG. 3.

This demonstrates that the addition of excessive sugar increases the calcite crystal form of calcium carbonate, making it unsuitable for the production of calcium carbonate including a large amount of vaterite crystal form which has excellent solubility and absorption rate.

FIG. 5 shows a SEM image of calcium carbonate particles produced when the amount of sugar added in the first step was 2.34 Mm. When the amount of added sugar was 2.34 Mm, the calcium carbonate particle size was about 0.68 to 0.69 µm, indicating that calcium carbonate having an ideally small size was produced, as shown in Table 2 above.

### Change of the Calcium Carbonate Depending on the Ultrasonic Intensity and Stirring Speed

In order to investigate the change of the calcium carbonate depending on the ultrasonic intensity and the stirring speed of the second step, an experiment was performed by adjusting the ultrasonic intensity and the stirring speed of the second step to the ranges of 0 to 70% and 0 to 600 rpm, respectively.

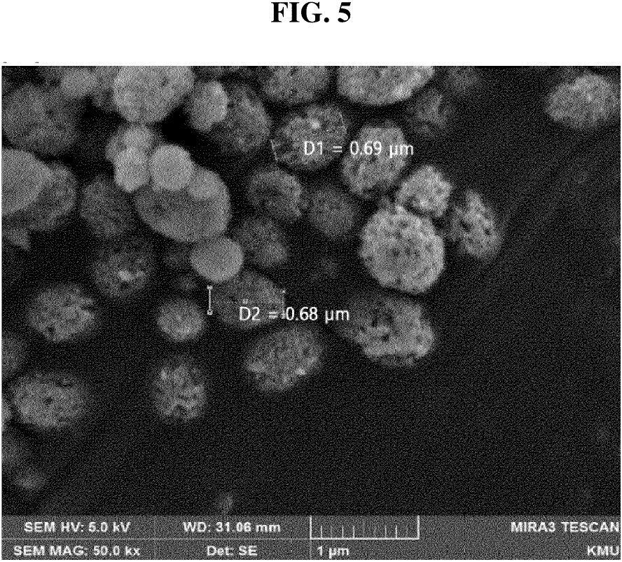

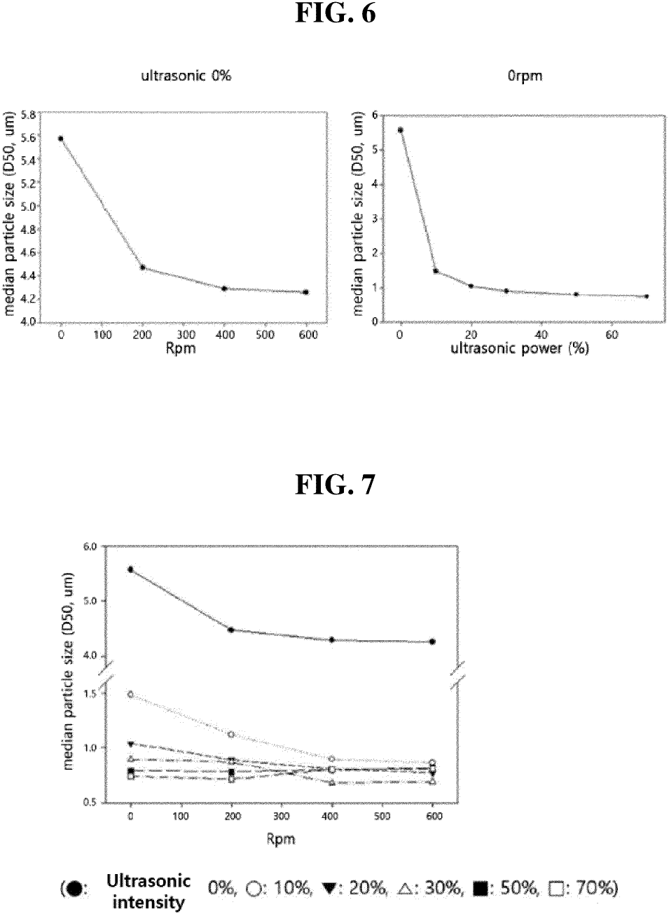

Table 3 below shows the particle size and shape of calcium carbonate produced under various ultrasonic intensity and stirring speed conditions. FIG. 6 shows graphs illustrating the change of the vaterite particle size with respect to the stirring speed and the change of the vaterite particle size with respect to the ultrasonic intensity. FIG. 7 shows a graph illustrating the change of the vaterite particle size with respect to the stirring speed and the ultrasonic intensity when stirring and ultrasonic waves were used at the same time.

Comparison of the particle size and shape of calcium carbonate particles produced under various ultrasonic intensity and stirring speed conditions

TABLE 3

| Ultrasonic intensity (%) | RPM | Median of particle size (D50, um) | Type of CaCO₃ | |
|---|---|---|---|---|
| | | | Vaterite (%) | Calcite (%) |
| 0 | 0 | 5.57 | 93 | 7 |
| | 200 | 4.47 | 95 | 5 |
| | 400 | 4.29 | 97 | 3 |
| | 600 | 4.26 | 97 | 3 |
| 10 | 0 | 1.48 | 100 | 0 |
| | 200 | 1.12 | | |
| | 400 | 0.897 | | |
| | 600 | 0.866 | | |
| 20 | 0 | 1.04 | 100 | 0 |
| | 200 | 0.892 | | |
| | 400 | 0.806 | | |
| | 600 | 0.775 | | |
| 30 | 0 | 0.897 | 100 | 0 |
| | 200 | 0.874 | | |
| | 400 | 0.683 | | |
| | 600 | 0.694 | | |
| 50 | 0 | 0.793 | 100 | 0 |
| | 200 | 0.783 | | |
| | 400 | 0.803 | | |
| | 600 | 0.817 | | |
| 70 | 0 | 0.743 | 100 | 0 |
| | 200 | 0.717 | | |
| | 400 | 0.804 | | |
| | 600 | 0.809 | | |

As shown in Table 3, FIG. 6, and FIG. 7 above, when the ultrasonic intensity and the stirring speed were 30% and 400 rpm, respectively, the particle size of the produced vaterite was the smallest as 683 nm. At a stirring speed of 200 rpm or lower, the stronger the ultrasonic intensity, the smaller the particle size. However, from a stirring speed higher than that, an offset effect with respect to the strong ultrasonic waves was generated. The vaterite particle size was simultaneously affected by stirring and ultrasonic waves. When the ultrasonic intensity was 10% and 20%, the smallest vaterite was formed at a stirring speed of 600 rpm. When the ultrasonic intensity was 30%, the vaterite particle size was the smallest at a stirring speed of 400 rpm. When the ultrasonic intensity was 70%, the vaterite particle size was the smallest at a stirring speed of 200 rpm. As previously stated, the smallest nano-sized vaterite was formed under the conditions of the ultrasonic intensity of 30% and the stirring speed of 400, at which the mutual offset effect was minimized and the advantages of both ultrasonic waves and stirring were maximized.

### Change of Particle Size and Shape of the Produced Calcium Carbonate Depending on the Sugar Content Without Applying Ultrasonic Waves

Item above showed that the ultrasonic intensity of the second stage affects the calcium carbonate. Therefore, in order to investigate the change in calcium carbonate depending on the sugar content without applying ultrasonic waves, an experiment was performed by setting the stirring speed to 200 rpm without using ultrasonic waves in the second step. A method for producing calcium carbonate to which the process of applying ultrasonic waves is not applied is described below.

After mixing and dissolving sucrose in 100 mL of seawater so that the solid-liquid ratio between sugar and seawater became 0 to 1:5000, the seawater in which sucrose was dissolved and the calcinated CaO were mixed so that the solid-liquid ratio became 1:50. The combinations of solutions used in this evaluation example were the same as those described in Table 1 above. Then, the resulting mixture was stirred at 25° C. at 200 rpm for 1 hour, and then filtered through a 0.45 µm membrane filter (MCE04547A, HYUNDAI Micro Co.).

The calcium eluate filtered through the process above was poured into a beaker and stirred at 200 rpm by using a stirrer (HS-30D, WISD), while 99.9% carbon dioxide was injected at a flow rate of 0.15 L/min by using a gas disperser (Sigma). A gas flow meter and a flow regulator (TSM-D220, MKP) were used to control the flow rate to be constant. The carbonation reaction was stopped by discontinuing the injection of carbon dioxide gas, and aging was performed by stirring at 200 rpm for 10 minutes. The generated solid was filtered through a 0.1 µm membrane filter (A010A047A, Toyo Roshi Kaisha) and dried at 60° C. for 4 hours.

Table 4 below shows the particle size and shape of the calcium carbonate produced in the absence of ultrasonic wave application.

Change in particle size and shape of the produced calcium carbonate according to the change in the amount of added sucrose

TABLE 4

| Elution conditions | | Results of carbonation | | |
|---|---|---|---|---|
| Amount of added sucrose (mM) | Calcium: sucrose molar ratio (mol:mol) | Median of calcium carbonate particle size (D₅₀ (µm) ) | Type of CaCO₃ (%) | |
| | | | Vaterite | Calcite |
| 0 | 0 | 4.09 | 70.5 | 29.5 |
| 0.58 | 1 : 0.01 | 4.12 | 82.1 | 17.9 |
| 1.17 | 1 : 0.01 | 4.03 | 85.4 | 14.6 |
| 2.34 | 1 : 0.02 | 3.57 | 90.2 | 9.8 |
| 4.67 | 1 : 0.05 | 3.43 | 91.2 | 8.8 |
| 9.35 | 1 : 0.09 | 3.22 | 92.7 | 7.3 |
| 18.70 | 1 : 0.16 | 3.12 | 93.2 | 6.8 |
| 37.51 | 1 : 0.26 | 2.57 | 94.5 | 5.5 |
| 75.02 | 1 : 0.47 | 2.41 | 94.6 | 5.4 |
| 107.48 | 1 : 0.67 | 2.65 | 72.4 | 27.6 |
| 150.04 | 1 : 0.92 | 3.15 | 70.2 | 29.8 |
| 214.99 | 1 : 1.20 | 3.28 | 82.5 | 17.5 |

As shown in Table 4 above, as the amount of added sucrose was increased, the vaterite content of the produced calcium carbonate tended to increase, and the particle size of the produced calcium carbonate gradually decreased and tended to increase from the time when the ratio of calcium to sucrose became approximately 2:1, that is, after the amount of added sucrose was 75.02 mM. In this case, the median of the calcium carbonate particle size was the smallest as 2.41 µm, and the vaterite content was also the highest as 94.6%. On the other hand, in the case of Table 4 where no ultrasonic wave was applied, 100% vaterite calcium carbonate was not formed, indicating that the application of ultrasonic waves is necessary to produce 100% vaterite calcium carbonate, which has a better effect.

Therefore, items (3) and (4) above showed that the ultrasonic intensity of the second step also significantly affects the production of 100% vaterite calcium carbonate.

### * Change of Calcium Carbonate Depending on the Aging Step

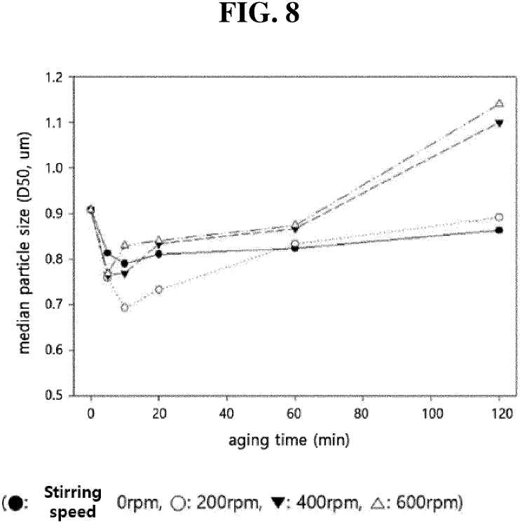

FIG. 8 shows a graph illustrating the particle size of calcium carbonate produced under different aging conditions after carbonation. As shown in FIG. 8, after the second step, when the calcium carbonate was left in the solution to which carbon dioxide injection was discontinued, recrystallization of vaterite did not occur under any conditions, and 100% vaterite form was maintained for 120 minutes. In addition, the particle size of vaterite was the smallest when stirring was performed at 200 rpm for 60 minutes or less, preferably for 2 to 20 minutes, and more preferably for 10 minutes, and the particle size was decreased by about 200 nm when the solution was filtered immediately without a step of stirring. Therefore, even when all conditions of the carbonation step are optimal, an appropriate aging step must be carried out in order to generate smaller nano-sized vaterite.

### 2. Analysis of Efficacy of Vaterite Calcium Carbonate

In order to investigate the efficacy of the calcium carbonate produced by the production method of the present invention in the body, the difference of the efficacy of calcium carbonate was analyzed by differently adjusting the particle size and crystalline form of calcium carbonate as shown in Table 5 below. Examples V1 to V4 used in the analysis below are examples of calcium carbonate of the vaterite crystalline form, wherein the calcium carbonate particle size of V1 was 9.18 µm, the calcium carbonate particle size of V2 was 4.17 µm, the calcium carbonate particle size of V3 was 1.33 µm, and the calcium carbonate particle size of V4 was 0.85 µm, as the calcium carbonate was adjusted to have different particle sizes. The particle size of the vaterite calcium carbonate claimed in the present invention is most similar to V4. In addition, examples C1 to C4 are examples of calcium carbonate of the calcite crystalline form for comparison with the calcium carbonate of the vaterite crystalline form. As in the case of vaterite, the calcium carbonate particle size of C1 was 11.4 um, the calcium carbonate particle size of C2 was 2.83 µm, the calcium carbonate particle size of C3 was 1.54 µm, and the calcium carbonate particle size of C4 was 0.657 µm. In addition, biomarkers that are related with the efficacy of calcium agent were selected, and an in vitro experiment was performed by measuring the calcium solubility and the cytotoxicity and ALP activity in osteoblast MG-63 to analyze examples of V1 to V4 and C1 to C4.

TABLE 5

| No. | Type of calcium carbonate | Calcium carbonate particle size (D50, µm) |
|---|---|---|
| V1 | Vaterite | 9.18 |
| V2 | Vaterite | 4.17 |
| V3 | Vaterite | 1.33 |
| V4 | Vaterite | 0.85 |
| C1 | Calcite | 11.4 |
| C2 | Calcite | 2.83 |
| C3 | Calcite | 1.54 |
| C4 | Calcite | 0.657 |

### ALP Activity Measurement

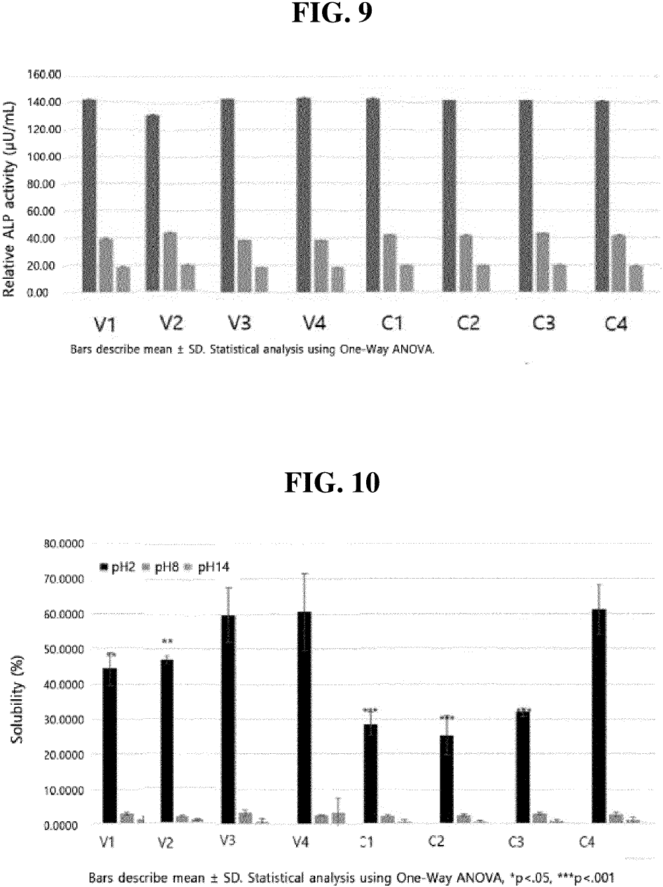

FIG. 9 shows a graph about the relative activity of ALP with respect to the calcium carbonate particle size depending on the amount of injected calcium carbonate.

Alkaline phosphatase (ALP) is the most commonly used bone formation marker in clinical practices and is a glycoprotein enzyme

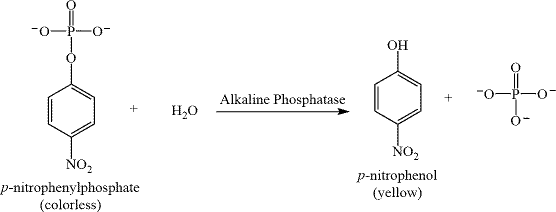

which is generated during osteoblastic bone formation and part of which is secreted into the blood. Therefore, when osteoblasts are actively accumulated in the bone matrix, the expression of ALP is increased, and its concentration is increased along with the increase of the bone activity.

In order to measure the relative activity of the ALP, the cells were treated with the calcium carbonate produced differently by the production method of the present invention at concentrations of 1, 5, and 10 mM, respectively. One-way ANOVA was used for the analysis after the measurement.

First, a 6-well plate in which MG-63 cells were cultured was left overnight to 3.5 × 10⁵ cells, treated with the test substances at different concentrations, and the cultured for 24 hours. The cells were washed twice with PBS, and were detached by using a cell scraper (SPL, 90030). After that, the cells were precipitated at 1,200 rpm for 1 minute, and the supernatant was removed. Thereafter, 100 µl of the ALP buffer included in the ALP kit (alkaline phosphatase assay kit, ab83369) was added and homogenized. The cell homogenate was centrifuged at 10,000 × g at 4° C. for 15 minutes to separate the supernatant, and then the intracellular ALP activity was measured by using an ALP kit through a microplate reader.

In all the examples of V1 to V4 and C1 to C4, it was confirmed that the relative ALP activity was increased as the concentration of the treated calcium carbonate was increased. At this time, the ALP was increased depending on the concentration of calcium carbonate. Therefore, it was confirmed that the treatment with the calcium carbonate of the present invention increases the ALP activity of cells.

### Solubility Measurement

FIG. 10 shows a graph about the solubility with respect to the calcium carbonate particle size depending on the pH of the surrounding environment.

In order to have excellent efficacy when a calcium agent is used as an oral calcium agent, the absorption rate must be good at a low pH, which is a gastric acid environment in the body. Therefore, to prove this, the solubility was measured in the environment of pH 2, pH 8, and pH 14 in respective examples. One-way ANOVA was used for the analysis after the measurement.

First, 0.5 mL of the sample, 0.5 mL of 10 mM calcium chloride, and 1.0 mL of 20 mM phosphate buffer (pH 8) were mixed, and then subject to a reaction at 37° C. for 2 hours. Thereafter, centrifugation was performed at 25° C. at 2,000 × g for 30 minutes. A calcium colorimetric analysis (OCPC method) was performed to measure absorbance at a wavelength of 575 nm, and based on the results, the calcium solubility was calculated by using the formula below.

$$\begin{matrix}\text{Calcium solubility} \left(\%\right) = \left(\text{calcium concentration in the supernatant} /\right) \\ \left(\text{total calcium concentration in the solution}\right) \times 100\end{matrix}$$

In all the examples, the solubility was significantly higher in an acidic environment of pH 2, and in particular, the highest solubility was found in V3 and V4 where the crystalline form was vaterite and the particle size was small. The results showed that the calcium carbonate produced through the present invention is easy to use as an oral calcium agent, because of its high solubility in the gastric acid environment.

As described above, the present invention has been mainly described with reference to preferable Examples, but those of ordinary skill in the art to which the present invention pertains may understand that the present invention may be variously modified and changed without departing from the principle and scope of the present invention as recited in the following claims.

1. A method for producing calcium carbonate, the method comprising:
a first step of eluting calcium by mixing calcined shells, seawater, and sugar; and
a second step of producing calcium carbonate by injecting carbon dioxide into the calcium eluate produced in the first step.

2. The method for producing calcium carbonate according to claim 1, wherein when the solid-liquid ratio of the sugar and seawater added in the first step is 1:80 (g:mL) or less, the vaterite content of calcium carbonate is 100%.

3. The method for producing calcium carbonate according to claim 1, wherein the amount of elution of calcium is increased in the first step.

4. The method for producing calcium carbonate according to claim 1, wherein the sugar is sucrose.

5. The method for producing calcium carbonate according to claim 1, wherein when the solid-liquid ratio of the sugar and seawater added in the first step is 1:5000 to 1:500 (g:mL), the vaterite crystal has a particle size of 600 to 800 nm.

6. The method for producing calcium carbonate according to claim 1, wherein the pH after the completion of the first step is 12.5 or higher.

7. The method for producing calcium carbonate according to claim 1, wherein the second step comprises applying ultrasonic waves to the solution into which carbon dioxide has been injected.

8. The method for producing calcium carbonate according to claim 1, wherein the production method further comprises a step of stirring the produced calcium carbonate at room temperature after the carbonation reaction of the second step.

9. The method for producing calcium carbonate according to claim 8, wherein the step of stirring is performed for 60 minutes or less.

10. The method for producing calcium carbonate according to claim 9, wherein the particle size of the calcium carbonate produced by the production method is in a range of 600 nm to 800 nm.

11. The method for producing calcium carbonate according to claim 1, wherein the calcium carbonate produced by the production method has porosity.

12. A calcium agent comprising vaterite-type calcium carbonate.

13. The calcium agent according to claim 12, wherein the calcium agent is produced by a method for producing calcium carbonate, the method comprising: a first step of eluting calcium by mixing calcined shells, seawater, and sugar; and a second step of producing calcium carbonate by injecting carbon dioxide into the calcium eluate produced in the first step.
